(19) United States
(12) Patent Application Publication
(10) Pub. No.: US 2005/0003342 A1
(43) Pub. Date: Jan. 6, 2005

(54) Viral marker

(75) Inventors: Nick Davis Poynter, Newmarket (GB); Josephine Nugent, Newmarket (GB); Ian Birch-Machin, Newmarket (GB); George P. Allen, Lexington, KY (US)

Correspondence Address: NIXON & VANDERHYE, PC, 1100 N GLEBE ROAD, 8TH FLOOR, ARLINGTON, VA, 22201-4714, US

(73) Assignee: Animal Health Trust, Newmarket (GB)

(21) Appl. No.: 10/626,832

(22) Filed: Jul. 25, 2003

Related U.S. Application Data
(60) Provisional application No. 60/398,576, filed on Jul. 26, 2002.

(51) Int. Cl. C12Q 1/70

(52) U.S. Cl. 435/5

(57) ABSTRACT

The invention relates to herpesviruses genetic markers, and provides methods and materials for sub-typing, classifying, identifying or monitoring an Equine herpesvirus type 1 (EHV-1) isolate, the method comprising use of genetic markers shown in Table 5 (preferably those shown in ORF68). In another aspect the invention provides methods and materials for assessing the virulence of herpesviruses comprising the use of genetic markers (e.g. within the herpesvirus DNA polymerase (ORF30 in EHV-1). In another aspect the invention provides vaccines based on the manipulation of the markers disclosed.

### TECHNICAL FIELD

This invention relates to genetic markers that correlate with virulence capacity for herpesviruses and methods and materials employing these. It further relates to processes for producing viruses having reduced virulence, and compositions based on these having protective effect.

### BACKGROUND

Equine herpesvirus type 1 (EHV-1) is a highly prevalent equine pathogen that can present a variety of clinical symptoms, ranging from respiratory distress to the induction of abortion and occasionally neurological damage resulting in paralysis [1-5].

Two field isolates of EHV-1 have been characterised which typify the phenotypic characteristics of virulent (isolate AB4) and avirulent (isolate V592) strains. AB4 was originally isolated from a case of paralytic disease [11] and V592 from a multi-case outbreak of abortion, during which no neurological disease was reported [12]. Whereas AB4 infection results in severe disease, including abortion and paralysis, V592 infection results in relatively minor disease, largely restricted to a short-lived fever and mild respiratory disease; it does not result in neurological damage[8, 13]. The sequence of AB4 has been published [19, 20].

In order to assess the relative importance of EHV-1 strain variation upon disease outcome, it is important to be able to discriminate between groups of inter-related strains.

Previous studies have utilised DNA restriction fragment length polymorphism (RFLP) to separate field isolates of EHV-1 into sub-groups according to characteristic restriction enzyme site changes and the presence of variable numbers of copies of short sequence repeats. These studies have demonstrated that there is a relatively low frequency of mutation for EHV-1 and have suggested that distinct strains of EHV-1 exist in the field [14-18]. However, the relative lack of variation of EHV-1 sequences between strains has resulted in insufficient RFLP variants being identified to enable detailed epidemiological studies. Furthermore, although such analyses are useful for tracing the genetic relatedness of strains, they only allow identification of those changes resulting in restriction fragment variation, rather than the majority of changes that do not affect restriction sites.

Nugent et al (2001), in a poster and abstract (“Comparison between a paralytic and non-paralytic strain of EHV-1. Identification of variable sequence markers and their application in sub-typing of field isolates”) presented at the 26^{th} International Herpesvirus Workshop July 28^{th}-August 3^{rd}, Regensburg, Germany (www.ihw2001.de) compared the sequences of paralytic and non-paralytic strains of EHV to try and identify variable sequence markers. One of these (‘ORF68 (SQ1)’) was used to group 70 field isolates into 5 specific groups, certain of which suggested geographical restriction. However no distinct pattern of disease severity emerged from the grouping.

Genetic markers which permit the classification of herpesviruses are useful in grouping and identifying isolates. Markers which are strongly predictive of the severity of disease which a herpesvirus is capable of causing are useful e.g. in assessing the virulence of isolates, and also in engineering vaccines, for example which are attenuated or have modulated immunogenicity, CTL response, or immunopathology.

It will be apparent from the foregoing that the identification of one or more genetic markers which permit the classification of herpesviruses, and especially markers which are strongly predictive of the severity of disease which a herpesvirus is capable of causing would be a contribution to the art.

### DISCLOSURE OF THE INVENTION

In order to identify the genetic differences underlying the observed differences in pathogenic potential of these strains, the present inventors determined the complete genomic sequence of V592. This was compared with the published sequence of AB4 [19, 20] to establish regions of genetic heterogeneity between these strains, and to characterise a group of loci having sequence variation for a panel of EHV-1 field isolates from Great Britain and the U.S.A. These loci may be used to classify EHV-1 field isolates into different groupings.

The inventors further identified single nucleotide polymorphisms (specifying amino acid coding changes) of the DNA polymerase (DNA pol, ORF30, in region ORF30-ml). As described below the markers were present in isolates from paralytic outbreaks and did not tend to associate with any of the other variable sequence markers tested, indicating that this specific gene is likely to be a critical determinant of EHV-1 virulence.

One of these ORF30-ml region markers (at amino acid position 752 in EHV-1) occurs at a conserved position of the herpesvirus DNA polymerase. An alignment of the selected region for certain alpha, beta, gamma and unclassified herpesviruses is shown in Table 4. The sequence of the virulent strain AB4 (D₇₅₂), and the majority of ‘paralytic’ isolates tested, conforms to all of the herpesviruses DNA pol sequences shown in Table 4 (with the exception of PRV, which has a conserved amino acid substitution (E) at this position). In contrast, the sequence of V592 and the majority of other ‘non-paralytic’ isolates, encode N rather than D at this position.

Another ORF30-ml region marker (variant D760-G) was found in two ‘paralytic’ isolates which encode N rather than D at position 752, and may be a further marker for isolates with paralytic potential.

Since the DNA polymerase gene is present in all herpesviruses characterised to date and in view of the high level of conservation described above, the ORF30-ml region marker results of the present invention have implications for other herpesviruses (especially alphaherpesviruses) in addition to EHV-1 in the assessment of virulence, and identification of further markers for the same.

Previously, single amino acid changes of the DNA polymerase have been noted in Herpes simplex virus type 1 (HSV-1) which have given an attenuated phenotype. For example Larder et al (1986, J gen Virol. 67: 2501-2506) showed an attenuated mutation at amino acid position 597. Pelosi et al (1998, Virology 252: 364-372) showed reduced pathogenicity for position 842. Apart from the fact that both of these mutations are quite distinct and distant from that characterized in the present invention, it is also notable that these mutations were drug induced (i.e. not naturally occurring) and attenuation was not demonstrated in a natural host or in a natural environment. Therefore these mutations have no relevance to natural-strain typing or assessment, and their potential or otherwise for use in vaccines is unknown. Likewise mutations proximal to the paralytic marker position (D₇₅₂) (e.g. in domain II) have been noted which influence antiviral drug sensitivity for herpes simplex virus, varicella zoster virus and human cytomegalovirus [29, 30, 31 and references therein]. Aspects of the present invention relate, inter alia, to the use of an ORF30-ml region marker, such as the EHV-1 ORF30 amino acid 752 or 760 marker (or a related or corresponding HV marker) as a diagnostic tool for assessment of herpesvirus isolates with differing capacity to induce disease. Other aspects relate to the herpesvirus-derived proteins and nucleic acids modified in respect of the marker or region surrounding it. The invention also concerns recombinant virus strains, live viral vaccines, methods for making the strains and vaccines, and methods for immunizing a host against a virus.

In one aspect the present invention provides a method for assessing the virulence of an Equine herpesvirus isolate, the method comprising use of a genetic marker. In preferred embodiments this is a nucleic acid ORF30-ml region marker, particularly a polymorphic marker. Preferred Equine herpesvirus isolates which may be assessed are type 1 (EHV-1) and type 4 (EHV-4). EHV-4 is highly homologous to EHV-1 and naturally occurring isolates thereof are associated with different severities of disease.

By “ORF30-ml region” is meant the region extending from nucleotide 2251 to 2310, using the EHV-1 V592 numbering given herein.

A further aspect of the present invention provides a method for assessing the virulence of a herpesvirus isolate, the method comprising use of a marker (particularly a polymorphic marker) corresponding to an EHV-1 DNA pol ORF30-ml region marker.

By “corresponding to” is meant having an equivalent position when sequences are aligned to maximize identity (for example as shown herein in Table 4). Those skilled in the art are well able to make such alignments to find corresponding positions by eye or using commercially available software.

The ‘virulence’ of a herpesvirus is a measure of the severity of the disease which it is capable of causing in a susceptible host. As those skilled in the art are aware, this is likely to be influenced by a number of factors, including the age and physical condition of the host, whether the infection is primary, secondary or a reactivation of latent virus, the immune status of the host etc. Thus virulence can not be an absolute measure, and the presence of any given marker does not mean that infection with that isolate inevitably results in clinical signs of disease. Nevertheless the methods of the invention can be used to compare the virulence of different strains, or predict the severity of disease which the virus is capable of causing in a given host, subject to correction or normalisation of other contributory or protective factors.

In preferred embodiments the method may in particular be used to assess “neurovirulence” by which is meant its potential to cause neurological damage e.g. paralytic disease. For example, where the virus is a strain of EHV-1, earlier studies of the pathogenesis of EHV-1 infection, in particular relating to induction of abortion and neurological damage, have demonstrated that virulent strains are ‘endotheliotropic’, displaying the ability to disseminate to and establish infection at vascular endothelial sites, in particular within the endometrium and CNS [6-10]. Thus the methods of the present invention may be utilised to assess the degree to which that virus is endotheliotropic.

Indeed the fact that there is such a strong association between herpesvirus sequence markers and paralytic disease suggests strongly that the viral genotype, rather than other environmental or host factors, is the predominant determinant of whether infection will result in paralytic or non-paralytic disease outbreaks.

As used in the present application, the term “marker” refers generally to the difference or differences between the nucleotide sequence of different groupings of herpesvirus. As described herein, markers may be associated with different strains and isolates, or with different levels of virulence. In certain embodiments the marker may be a virulence marker, which is a nucleotide sequence difference between a virulent form of a herpesvirus and the nucleotide sequence of a corresponding strain having reduced virulence. As discussed below, the marker can be a single difference (one point difference) in a nucleotide sequence or differences in more than one nucleotide, wherein the different nucleotides are located in close proximity to each other.

Thus in other aspects the method provides for classifying a herpesvirus isolate in terms of virulence described above, the method comprising use of a marker as discussed herein.

In one preferred form the method relates to the EHV-1 ORF30-ml region marker at amino acid position 752 (based on the V592 numbering—see SEQ. ANNEX 1 and SEQ. ANNEX 3b) or one which corresponds to this when sequences are aligned as shown herein (see Table 4).

In another preferred form the method relates to the EHV-1 ORF30-ml region marker at amino acid position 760 or one which corresponds to this when sequences are aligned as shown herein (see Table 4).

Where amino acid or nucleotide positions are discussed hereinafter with reference to EHV-1, it will be understood that these discussions apply also to the corresponding position in other herpesvirus DNA polymerase sequences.

A comparison between herpesvirus DNA polymerase sequences and those of other related polymerases (from organisms as diverse as bacteriophage and mammals) has revealed a number of conserved domains [27, 28]. The ORF30-ml region lies between the conserved domains designated II and VI, which comprise core, catalytic regions essential for DNA polymerase activity. Thus, although this position is not within one of the domains known to be critical for function, it does lie within the core, catalytic region of the enzyme and it is therefore likely that coding change results in a change in functional properties of the enzyme and therefore plays a direct role in the aetiology of disease. However, irrespective of the precise underlying mechanism, the marker has utility in the methods described herein. Sequence variation at this marker position has not previously been noted for any other herpesvirus where multiple isolates have been analysed (e.g. human cytomegalovirus [29]).

Preferably the presence of an acidic amino acid at position 752 (e.g. glutamic acid ‘E’, or more preferably aspartic acid ‘D’) is correlated with higher virulence of the herpesvirus.

Preferably the presence of a non-acidic amino acid at position 760 (e.g. glycine ‘G’)is correlated with higher virulence of the herpesvirus.

In preferred embodiments the nucleotide sequence (codon) encoding the amino acid will be assessed.

The invention thus employs the identity of a codon at nucleotide positions 2254-2256 or 2278-2280 (based on the EHV-1 V592 numbering—see SEQ. ANNEX 1 and SEQ. ANNEX 3a) or one which corresponds to this when sequences are aligned.

Thus, preferably the presence of an ‘G’ at position 2254 is correlated with higher virulence of the herpesvirus.

Thus, preferably the presence of an ‘G’ at position 2279 is correlated with higher virulence of the herpesvirus.

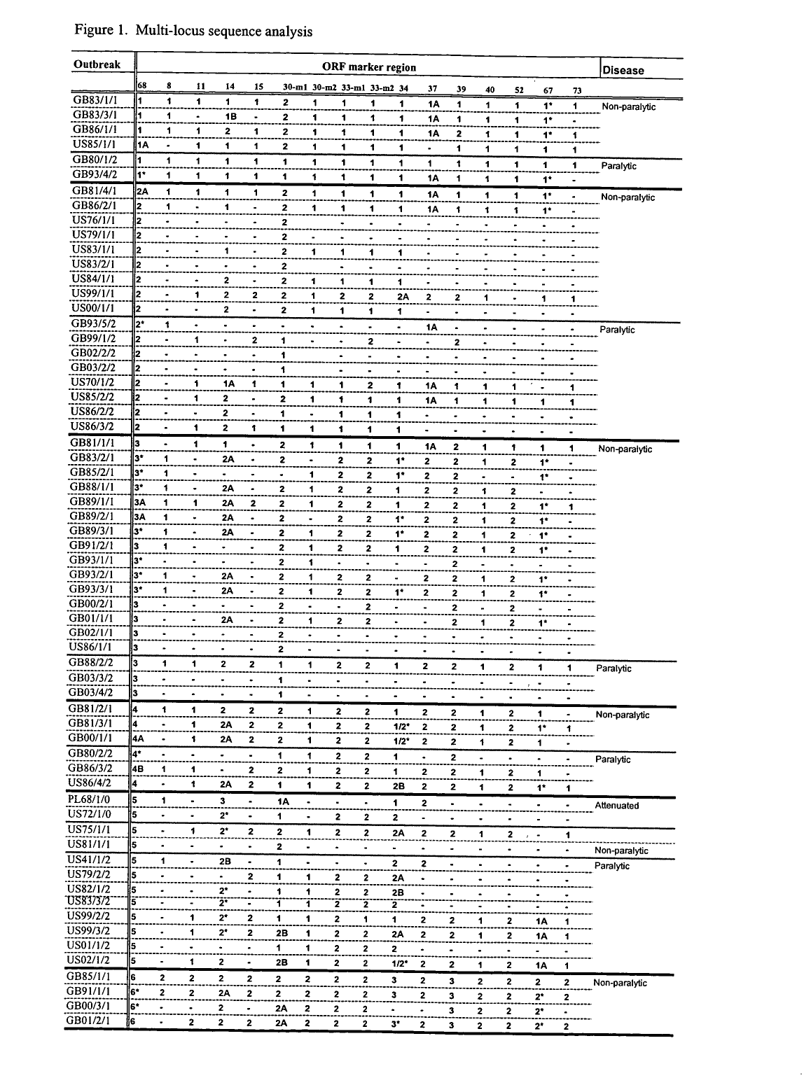

Regarding marker G₂₂₅₄, this shows an especially strong predictive value as a marker of isolates capable of causing paralytic disease in EHV-1. The fact that it does not co-segregate with the other markers tested (although the majority of these other markers do tend to co-segregate with each other according to the groupings described in FIG. 1) suggests that it may arise ‘spontaneously’ rather than being inherited along with other markers for a specific group of related EHV-1 strains. In particular, it is significant that ORF30 G₂₂₅₄ does not co-segregate with another ORF30 marker (G-A₂₉₆₈), which is separated by a distance of only 714 nucleotides.

For brevity hereinafter, the term “ORF30-ml region virulence marker” may be used (except when context demands otherwise) to describe not only markers from EHV-1 such as those found at nucleotide positions 2254-2256 (preferably 2254 discussed above) positions 2278-2280 (preferably 2279 discussed above) but also virulence markers from corresponding DNA pol regions from other herpesviruses, especially alphaherpesviruses (which may or may not be termed “ORF30” therein, see Table 4). Likewise where positions are cited (amino acid or nucleotide e.g. 752 or 760, or 2254 or 2279) in relation to EHV-1 markers, the corresponding positions from other herpesviruses, especially alphaherpesviruses, are included. Likewise, discussions of embodiments with respect to EHV apply to EHV-1 and EHV-4, and correspondingly to other herpesviruses.

The method of the aspects above may comprise:
(i) providing a sample of nucleic acid from the herpesvirus isolate, and,
(ii) establishing the presence or identity of one or more DNA pol (ORF30) markers in the nucleic acid sample, preferably an ORF30-ml region virulence marker.

Preferred methods for detecting and determining markers such as SNP markers are described in more detail hereinafter.

As will be understood by those skilled in the art, where the term ‘isolate’ is used this should not be taken as requiring that the virus be in pure form. For examples the virus may be present in a sample e.g. an environmental or biopsy sample. The method may be preceded by a culturing step in order to cause or permit replication of herpesviruses in a sample. For example, for EHV, equine fibroblasts may be infected and incubated (37° C.) until 50-100% c.p.e. had developed. Cells can then be pelleted by centrifugation, washed with TE buffer and resuspended in Proteinase K/SDS solution 0.1 mg/ml Proteinase K, 0.5% SDS). Following 1-2 hours digestion, DNA can be prepared by phenol/chloroform extraction followed by ethanol precipitation. Purified DNA is then re-dissolved in TE buffer.

Equally the isolate may be latent i.e. nucleic acid harboured within a host cell. Thus in one embodiment, the methods of the invention are used to characterise herpesvirus (e.g. EHV) strains harboured latently e.g. via PCR amplification and sequencing of the ORF30-ml region from peripheral blood mononuclear cells (a site of EHV-1 latency). For example methods described by Welch et al (1992, J Gen Virol 73:261-268) could be used, or methods analogous to these.

Determination of whether horses are infected with potentially paralytic strains of EHV-1 will be useful in refining management procedures, for example employing appropriate isolation procedures to limit the risk of such animals undergoing reactivation of the latent virus and hence transmitting ‘paralytic’ EHV strains to susceptible animals (in particular pregnant mares).

The identification of a marker linked to paralytic disease has important implications for current and future live HV vaccines. In view of the risk of reversion to virulence of a live attenuated strain, or recombination between an attenuated vaccine strain and virulent field strain, vaccine strains can be screened for the presence of markers disclosed herein.

The prerequisite for a useful HV mutant vaccine is that the mutation is incorporated in a permissive position or region of the HV genome, i.e. a position or region which can be used for the incorporation of the mutation without disrupting essential functions of HV such as those necessary for infection or replication.

Preferred live vaccines of the present invention may be those which that are engineered in the light of the disclosure herein to carry reduced-virulence ORF30-ml region virulence markers (e.g. the marker sequence of V592, wherein position 752 is a non-acidic side chain amino acid i.e. one which does not have a negative charge at neutral pH, and position 760 is an acidic side chain amino acid).

Thus the present invention relates to recombinant herpesvirus strains, live viral vaccines incorporating such strains, methods for making such strains and vaccines, and methods for immunizing a host against herpesvirus infection using the vaccines wherein the viral DNA encodes a gene product modified in respect of one or more ORF30-ml region virulence markers.

For example the invention provides an EHV vaccine based on an EHV genome consisting essentially of an EHV which has reduced virulence as a result of its DNA POL (ORF30)gene having at least one, preferably two, attenuating mutations (e.g. substitution or deletion) at ORF30-ml region virulence marker sites. The genome may be otherwise virulent, or may include other attenuating modifications.

Alternatively, methods of preparing vaccines according to the present invention may include making a modification within about 1,2,3,4,5,10 or 15 amino acids or codons of an ORF30-ml region virulence marker disclosed herein.

A method for preparing a vaccine according to the present invention may include the steps of:
(i) providing nucleic acid from an herpesvirus genome,
(ii) modifying the DNA POL gene of the herpesvirus to reduce the virulence of the gene product of said gene, said modification being within an ORF30-ml region virulence marker or the range defined above of that marker; and,
(iii) combining the modified virus encoded by the genome with a pharmaceutically acceptable diluent, adjuvant, or carrier.

Preferably the modification is selected from the group consisting of insertions, substitutions, and deletion at the position 752 or 760 markers described herein, most preferably the 752 marker. Preferably the modifying step comprises the step of substituting one or more nucleotides within the marker. Recombinant HV (for example) may be prepared by recombination between modified plasmid and full length viral DNA (following cotransfection) or by manipulation of a full length infectious clone of HV (e.g. a BAC clone)—see e.g. Seyboldt et al (2000) Virology 278: 477-489, or Rudolph et al (2002) J Vet Med B Infect Dis Vet Public Health 49: 31-36.

Preferably the herpesvirus is EHV (e.g. EHV-1). For the preparation of a live vaccine the EHV mutant according to the present invention can be grown on a cell culture of equine origin or on cells from other species. The viruses thus grown can be harvested by collecting the tissue cell culture fluids and/or cells.

The live vaccine may be prepared in the form of a suspension or may be lyophilized. In addition to an immunogenically effective amount of the HV mutant vaccine may contain a pharmaceutically acceptable carrier or diluent, as described below.

HV mutants according to the invention can also be used to prepare an inactivated vaccine.

One explanation for the increased virulence of herpesviruses having the AB4 sequence is that immune responses against e.g. EHV-1 encoding the DNA polymerase D₇₅₂ marker may be modified compared with those to EHV-1 carrying the N₇₅₂ marker, either resulting in less efficient clearance of virus infected cells or more severe immunopathology at the sites of virus infection.

For example the region of DNA polymerase including amino acid position 752 and\or 760 may be a CTL epitope, and variations at these positions may modify induction of, or susceptibility to, CTL responses.

For example if the majority of EHV-1 isolates in the field carry the N₇₅₂ marker, then horses will tend to be naturally primed (via infection) to the ‘non-paralytic’ CTL epitope and hence may be less able to clear infection from those isolates carrying the ‘paralytic’ marker, due to the lack of CTLs primed against the ‘paralytic’ epitope.

If this is the case, then it may be desired to improve protection against paralytic EHV-1 disease by specific vaccination designed to prime CTL responses against the DNA pol epitope found in ‘paralytic’ strains. This is achieved by incorporation of the DNA pol sequence, or a region comprising the minimal CTL epitope including amino acid position 752 (from AB4) and\or 760 (from T937 or T949, isolates from the US99/3/2 and US02/1/2 outbreaks respectively—see FIG. 1) or both, in a vaccine formulation capable of stimulating CTL responses (e.g. delivery by ISCOMs, plasmid DNA or recombinant virus vaccines).

As is well known to those skilled in the art, CTL inducing peptides are typically small peptides that are derived from selected epitopic regions of target antigens associated with an effective CTL response to the disease of interest. Thus, by “CTL inducing peptide” or “CTL peptide” of the present invention is meant a chain of at least four amino acid residues, preferably at least six, more preferably eight to ten, sometimes eleven to fourteen residues, and usually fewer than about thirty residues, more usually fewer than about twenty-five, and preferably fewer than fifteen, e.g. eight to fourteen amino acid residues derived from selected epitopic regions of the virulent marker sequence.

The precise size of an optimum epitope including the marker can be determined by assessing its ability to stimulate CTL responses against EHV-1 infected target cells or to serve as a target for CTLs naturally primed by EHV-1 infection. Optionally comparisons can be made with a ‘non-paralytic’ sequence exemplified by V592.

Thus the present invention provides vaccines (e.g. attenuated in respect of the markers disclosed herein or CTL-inducing vaccines) optionally accompanied by a pharmaceutically acceptable diluent, adjuvant or carrier as described above. Thus a process for producing the same by combining said vaccines with the pharmaceutically acceptable ingredients are also provided.

The prophylactic and therapeutic materials discussed above and based on the markers disclosed herein may be formulated with appropriate carriers. For example dispersions can be prepared in glycerol, liquid polyethylene glycols, and mixtures thereof, and in oils. Under ordinary conditions of storage and use, these preparations can contain a preservative to prevent the growth of microorganisms. The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion and by the use of surfactants. The preventions of the action of microorganisms can be brought about by various antibacterial and antifungal agents, for example, parabens, chlorobutanol, phenol, sorbic acid, thimerosal, and the like. In many cases it may be preferable to include isotonic agents, for example, sugars or sodium chloride.

Prolonged absorption of the injectable compositions can be brought about by the use in the compositions of agents delaying absorption, for example, aluminum monostearate and gelatin. Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yield a powder of the active ingredient plus any additional desired ingredient from previously sterile-filtered solution thereof.

Thus examples of pharmaceutically acceptable carriers or diluents useful in the present invention include stabilizers such as SPGA, carbohydrates (e.g. sorbitol, mannitol, starch, sucrose, glucose, dextran), proteins such as albumin or casein, protein containing agents such as bovine serum or skimmed milk and buffers (e.g. phosphate buffer). Optionally, one or more compounds having adjuvant activity may be added to the vaccine. Suitable adjuvants are for example aluminium hydroxide, phosphate or oxide, oil-emulsions. Adjuvants contemplated herein include resorcinols, non-ionic surfactants such as polyoxyethylene oleyl ether and n-hexadecyl polyethylene ether.

As used herein “pharmaceutically acceptable carrier” includes any and all of these solvents, dispersion media, coatings, antibacterial and antifungal agents, isotonic and adsorption delaying agents, and the like described above. Supplementary active ingredients can also be incorporated into the compositions.

The active compounds for vaccination or passive immunization may be administered in a convenient manner such as by intravenous (where water soluble), intramuscular, subcutaneous, intranasal, or intradermal routes. Intramuscular administration is a preferred method of administration but other methods are also contemplated by the present invention. The active compounds may also be administered parenterally or intraperitoneally.

It is especially advantageous to formulate parenteral compositions in dosage unit form for ease of administration and uniformity of dosage. Dosage unit form as used herein refers to physically discrete units suited as unitary dosages for the mammalian subjects to the treated; each unit containing a predetermined quantity of the active material calculated to produce the desired therapeutic effect in association with the required pharmaceutical carrier. The specification for the novel dosage unit forms of the invention are dictated by and directly depend on (a) the unique characteristics of the active material and the particular therapeutic effect to be achieved, and (b) the limitations inherent in the art of compounding such active material for the treatment of disease.

The principal active ingredient is compounded for convenient and effective administration in effective amounts with a suitable pharmaceutically acceptable carrier in dosage unit form as hereinbefore disclosed. A unit dosage form can, for example, contain the principal active compound in amounts ranging from 0.5 μg to about 2000 mg. Expressed in proportions, the active compound is generally present in from about 10 μg to about 2000 mg/ml of carrier. In the case of compositions containing supplementary active ingredients, the dosages are determined by reference to the usual dose and manner of administration of the said ingredients.

The useful dosage to be administered will vary depending on the age and weight of the animal, and mode of administration. A suitable dosage can range for example from 10³ to 10⁸ TCID₅₀ (preferably around 10⁶ TCID₅₀) of the EHV mutant per horse.

The present invention further provides methods for immunizing a host against a herpesvirus which include a step of inoculating the host with an immunity-inducing dose of a vaccine as described above. For example, a live EHV mutant according to the present invention can be used to vaccinate horses.

Also provided are vaccines for use in such methods, and vaccines (or modified herpesviral genomes) for use in the preparation of such vaccines.

Although not wishing to be bound by any particular theory the ORF30 coding change corresponding to the ORF30-ml region virulence marker s described herein may result in a change in functional properties of the DNA polymerase which plays a direct role in the aetiology of paralytic disease. Such functional differences in ORF30 may affect replication, for example in specific cell types or in the context of tissues in vivo.

In particular, ORF30 may play a role during establishment of, or reactivation from, latency. Thus, establishment of latency may result following ‘abortive’ replication, if DNA pol activity is insufficient to promote full late gene expression prior to the genome entering a quiescent state; conversely, during the early stages of reactivation, DNA pol activity may be critical in triggering full lytic cycle gene expression. An effect upon latency/reactivation would be consistent with the apparent ‘independence’ of the ORF30-ml region virulence markers compared with the other markers tested, if isolates carrying this marker are attenuated for establishment of, or reactivation from, latency.

Either cell-specific replication differences or modulation of latency/reactivation may be relevant to the efficiency with which EHV-1 infected lymphocytes (a major source of infectious virus carried via cell-associated viraemia and a major site of EHV-1 latency) transfer virus to vascular endothelial cells of blood vessels serving the CNS, infection of which is a consistent feature of paralytic disease. If the ‘paralytic’ marker enables virus strains to replicate more rapidly in critical cell types (eg. respiratory epithelia, lymphocytes or endothelial cells), then this may result in increased viral load being delivered to, or replicating in, the vascular endothelia. Similarly, if strains carrying the paralytic marker tend to establish lytic, rather than latent, infection of lymphocytes, then this may result in a higher proportion of infected lymphocytes undergoing active virus replication and hence delivering infectious virus to the vascular endothelia with higher efficiency.

The invention also provides materials which may be used in the methods disclosed herein. These include isolated nucleic acid molecules consisting of the DNA sequence of EHV-1 (strain V592) ORF30 shown in SEQ. ANNEX 3, which sequence comprises a mutation which reduces the virulence of the gene product.

Also provided are isolated peptides comprising, or consisting of, or consisting essentially of, a contiguous portion of at least 10, 15, 20, 30, 40, or 50 amino acids of the amino acid sequence of an EHV-1 strain (preferably strain V592) ORF30 shown in SEQ. ANNEX 3, wherein the portion includes position 752 or 760 of SEQ. ANNEX 3.

Such peptides and nucleic acids according to the present invention may be provided isolated and/or purified from their natural environment, in substantially pure or homogeneous form, or free or substantially free of other nucleic acids of the species of origin. Where used herein, the term “isolated” encompasses all of these possibilities.

The invention further provides oligonucleotides for use in probing or amplification reactions which are selective for the markers described herein.

An oligonucleotide for use in nucleic acid amplification may be about 30 or fewer nucleotides in length (e.g. 18, 21 or 24). Generally specific primers are upwards of 14 nucleotides in length, but need not be than 18-20. The oligonucleotides bind to regions in close proximity to the marker under investigation. The region amplified by PCR technology will usually have a length of about 60 to 600 nucleotides. Those skilled in the art are well versed in the design of primers for use processes such as PCR. Various techniques for synthesizing oligonucleotide primers are well known in the art, including phosphotriester and phosphodiester synthesis methods.

Preferred primers for the amplification of regions of variable sequence for several ORFs are shown in SEQ. ANNEX 2. Of these, the primers for amplification of the ORF30-ml marker region (ORF30f, ORF30r) and of the ORF68 marker region (ORF68f, ORF68r) are particularly preferred.

Primers complementary to those disclosed herein are also embraced by the present invention. By the term “complementarity” is meant a sufficient number in the oligonucleotide of complementary base pairs in its sequence to interact specifically (hybridize) with the target nucleic acid sequence of the herpesvirus to be amplified or detected. As known to those skilled in the art, a very high degree of complementarity is needed for specificity and sensitivity involving hybridization, although it need not be 100%. Thus, for example, an oligonucleotide which is identical in nucleotide sequence to an oligonucleotide disclosed herein, except for one base change or substitution, may function equivalently to the disclosed oligonucleotides.

Compositions of such oligonucleotides are also provided. By the term “composition” is meant a combination of elements which may include one or more of the following: the reaction buffer for the respective method of enzymatic amplification, plus one or more oligonucleotides specific for the herpesvirus marker labeled with a detectable moiety.

Nucleic acid for use in the methods of the present invention, such as an oligonucleotide probe and/or pair of amplification primers, may be provided in isolated form and may be part of a kit, e.g. in a suitable container such as a vial in which the contents are protected from the external environment. The kit may include instructions for use of the nucleic acid, e.g. in PCR and/or a method for determining the presence of nucleic acid of interest in a test sample. A kit wherein the nucleic acid is intended for use in PCR may include one or more other reagents required for the reaction, such as polymerase, nucleosides, buffer solution etc. The nucleic acid may be labelled. A kit for use in determining the presence or absence of nucleic acid of interest may include one or more articles and/or reagents for performance of the method, such as means for providing the test sample itself

The present invention is particularly directed to a kit for typing or assessing the virulence of herpesviruses. When the diagnostic test is based on polymerase chain reaction (PCR) technology, the kit will contain at least a first oligonucleotide which selectively binds to DNA on the 3′ side of the marker and a second oligonucleotide which selectively binds to DNA on the 5′ side of the marker. More specifically two oligonucleotides flank the marker sequence, bind to the opposite strands of DNA and serve as primers for PCR leading to amplification of marker-containing DNA sequence.

Identification of multiple positions of sequence variation between EHV-1 (V592) and strain (AB4) has demonstrated their usefulness as a method for sub-typing EHV-1 isolates recovered from the field, via multi-locus sequence typing. These methods will be useful therefore in tracing the transmission of virus strains between outbreaks and, in horse populations where live EHV-1 vaccination is employed, determining whether outbreaks result from reversion to virulence of the vaccine strain.

Thus the use of any marker based upon ORF sequences which vary between AB4 and V592 disclosed in Table 2 for detecting the presence of, classifying, grouping, identifying or monitoring an EHV isolate forms one aspect of the present invention. Nucleotide sequence variation for selected marker regions, including positions of nucleotide polymorphism in addition to those noted between AB4 and V592, are disclosed in Table 5.

A preferred marker is that which appears in ORF68. Polymorphisms within this gene noted in Table 5 are preferred e.g. at nucleotide positions (numbered according ot the AB4 ORF68 sequence) 336, 344, 629, 710, 713, 719, 731-740, 755. In preferred embodiments the marker is used to classify the isolate into one of the 6 groups shown in FIG. 1. As described in Example 4 below, the ORF68 region permits the identification of six major, distinct, groups of related strains (on the basis of ORF68 sequences and supported by other markers tested), isolated from outbreaks that have occurred over the course of 20-30 years.

Some preferred techniques for use in the methods of the present invention will now be discussed in more detail.

General methods for assessment of polymorphisms are reviewed by Schafer and Hawkins, (Nature Biotechnology (1998)16, 33-39, and references referred to therein) and include: oligonucleotide probing, amplification using PCR, denaturing gradient gel electrophoresis, RNase cleavage, chemical cleavage of mismatch, T4 endonuclease VII cleavage, multiphoton detection, cleavase fragment length polymorphism, E. coli mismatch repair enzymes, denaturing high performance liquid chromatography, (MALDI-TOF) mass spectrometry, analysing the melting characteristics for double stranded DNA fragments as described by Akey et al (2001) Biotechniques 30; 358-367. These references, inasmuch as they be used in the performance of the present invention by those skilled in the art, are specifically incorporated herein by reference.

The assessment of polymorphisms may be carried out on a DNA microchip, if appropriate. One example of such a microchip system may involve the synthesis of microarrays of oligonucleotides on a glass support. Fluorescently-labelled PCR products may then be hybridised to the oligonucleotide array and sequence specific hybridisation may be detected by scanning confocal microscopy and analysed automatically (see Marshall & Hodgson (1998) Nature Biotechnology 16: 27-31, for a review).

Use of Nucleic Acid Probes

The method of assessment of the polymorphism may comprise determining the binding of an oligonucleotide probe to the nucleic acid sample. The probe may comprise a nucleic acid sequence which binds specifically to a particular marker polymorphism (e.g. G) and does not bind specifically to other possible base identities at the polymorphism (e.g. A). Where the nucleic acid is double-stranded DNA, hybridisation will generally be preceded by denaturation to produce single-stranded DNA. A screening procedure, chosen from the many available to those skilled in the art, is used to identify successful hybridisation events and isolated hybridised nucleic acid.

Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter. Labelled probe may be hybridised to the DNA fragments on the filter and binding determined.

Where the term “label” or “labelled” is used herein this refers to a detectable molecule which is incorporated indirectly or directly into an oligonucleotide, wherein the label molecule facilitates the detection of the oligonucleotide. Methods of producing labelled probes (or primers—see below) are well known to those skilled on the art (See for example, Molecular Cloning, a laboratory manual: editors Sambrook, Fritsch, Maniatis; Cold Spring Harbor Laboratory Press, 1989; BioTechniques “Producing single-stranded DNA probes with the Taq DNA polymerase: a high yield protocol,” 10:36, 1991). Alternatively, the detectable moiety may be incorporated directly or indirectly such as, for example, by biotinylating the 5′ aminogroup of the oligonucleotide with sulfo-NHS-biotin. Other label molecules, known to those skilled in the art as being useful for detection, include radioactively, fluorescently or enzymatically labelled molecules. Various fluorescent molecules are known in the art which are suitable for use to label a nucleic acid substrate for the method of the present invention. Fluorescent molecules used as labels may include amine-reactive molecules which are reactive to end terminal amines of the substrate; sulfonyl chlorides which are conjugated to the substrate through amine residues; and the like. Depending on the fluorescent molecule used, incorporating the substrate with the fluorescent molecule label include attachment by covalent or noncovalent means. The protocol for such incorporation may vary depending upon the fluorescent molecule used. Such protocols are known in the art for the respective fluorescent molecule.

Binding of a probe to target nucleic acid (e.g. DNA) may be measured using any of a variety of techniques at the disposal of those skilled in the art.

Polymorphisms may be detected by contacting the sample with one or more labelled nucleic acid reagents including recombinant DNA molecules, cloned genes or degenerate variants thereof under conditions favorable for the specific annealing of these reagents to their complementary sequences within the relevant gene. Preferably, the lengths of these nucleic acid reagents are at least 15 to 30 nucleotides. After incubation, all non-annealed nucleic acids are removed from the nucleic acid:gene hybrid. The presence of nucleic acids that have hybridized, if any such molecules exist, is then detected. Using such a detection scheme, the nucleic acid from the cell type or tissue of interest can be immobilized, for example, to a solid support such as a membrane, or a plastic surface such as that on a microtitre plate or polystyrene beads. In this case, after incubation, non-annealed, labeled nucleic acid reagents are easily removed. Detection of the remaining, annealed, labeled nucleic acid reagents is accomplished using standard techniques well-known to those in the art. The gene sequences to which the nucleic acid reagents have annealed can be compared to the annealing pattern expected from a normal gene sequence in order to determine whether a gene mutation is present.

Approaches which rely on hybridisation between a probe and test nucleic acid and subsequent detection of a mismatch may be employed. Under appropriate conditions (temperature, pH etc.), an oligonucleotide probe will hybridise with a sequence which is not entirely complementary. The degree of base-pairing between the two molecules will be sufficient for them to anneal despite a mis-match. Various approaches are well known in the art for detecting the presence of a mis-match between two annealing nucleic acid molecules. For instance, RN'ase A cleaves at the site of a mis-match. Cleavage can be detected by electrophoresing test nucleic acid to which the relevant probe or probe has annealed and looking for smaller molecules (i.e. molecules with higher electrophoretic mobility) than the full length probe/test hybrid. Other approaches rely on the use of enzymes such as resolvases or endonucleases.

Thus, an oligonucleotide probe that has the sequence of a region of the marker described herein (either sense or anti-sense strand) may be annealed to test nucleic acid and the presence or absence of a mis-match determined. Detection of the presence of a mis-match may indicate the presence in the test nucleic acid of a mutation associated with the trait.

Those skilled in the art are well able to employ suitable conditions of the desired stringency for selective hybridisation, taking into account factors such as oligonucleotide length and base composition, temperature and so on.

Suitable selective hybridisation conditions for oligonucleotides of 17 to 30 bases include hybridization overnight at 42° C. in 6× SSC and washing in 6× SSC at a series of increasing temperatures from 42° C. to 65° C. One common formula for calculating the stringency conditions required to achieve hybridization between nucleic acid molecules of a specified sequence homology is (Sambrook et al., 1989): Tₘ=81.5° C.+16.6Log [Na+]+0.41(% G+C)−0.63(% formamide)−600/#bp in duplex. Other suitable conditions and protocols are described in Molecular Cloning: a Laboratory Manual: 2nd edition, Sambrook et al., 1989, Cold Spring Harbor Laboratory Press and Current Protocols in Molecular Biology, Ausubel et al. eds., John Wiley & Sons, 1992.

Amplification-Based Methods

The hybridisation of such a probe may be part of a PCR or other amplification procedure. Accordingly, in one embodiment the method of assessing the polymorphism includes the step of amplifying a portion of the ORF30 gene or other gene containing the marker of interest (e.g. an ORF30-ml region virulence marker).

The assessment of the polymorphism in the amplification product may then be carried out by any suitable method, e.g., as described herein. An example of such a method is a combination of PCR and low stringency hybridisation with a suitable probe. Unless stated otherwise, the methods of assessing the polymorphism described herein may be performed on a genomic DNA sample, or on an amplification product thereof.

Where the method involves PCR, or other amplification procedure, any suitable PCR primers may be used. Example primers are described herein. For example primers are shown in SEQ. ANNEX 2. Preferred primers are any of those listed as ORF30f, ORF30r, ORF68f and ORF68r.

Suitable polymerase chain reaction (PCR) methods are reviewed, for instance, in “PCR protocols; A Guide to Methods and Applications”, Eds. Innis et al, 1990, Academic Press, New York, Mullis et al, Cold Spring Harbor Symp. Quant. Biol., 51:263, (1987), Ehrlich (ed), PCR technology, Stockton Press, N.Y., 1989, and Ehrlich et al, Science, 252:1643-1650, (1991)). PCR comprises steps of denaturation of template nucleic acid (if double-stranded), annealing of primer to target, and polymerisation. An amplification method may be a method other than PCR. Such methods include strand displacement activation, the QB replicase system, the repair chain reaction, the ligase chain reaction, rolling circle amplification and ligation activated transcription. For convenience, and because it is generally preferred, the term PCR is used herein in contexts where other nucleic acid amplification techniques may be applied by those skilled in the art. Unless the context requires otherwise, reference to PCR should be taken to cover use of any suitable nucleic amplification reaction available in the art.

A preferred method for detecting the presence or absence of EHV-1 carrying the specified marker in a sample comprises the steps of:
(a) lysing the cells in a sample to release nucleic acid molecules;
(b) contacting the nucleic acid molecules with oligonucleotide primers as described herein under suitable conditions permitting hybridization of the oligonucleotides to the nucleic acid molecules;
(c) enzymatically amplifying a specific region of the nucleic acid molecules comprising the marker sequences using said oligonucleotides as primers;
(d) determining the identity of the marker in the amplified sequences
   Sequencing

The polymorphism may be assessed or confirmed by nucleotide sequencing of a nucleic acid sample to determine the identity of a polymorphic allele. The identity may be determined by comparison of the nucleotide sequence obtained with a sequence shown herein.

Preferred sequencing primers include any of those shown in SEQ. ANNEX 2, particularly ORF30s, ORF68s1, ORF68s2 and ORF68s3.

Mobility Based Methods

The assessment of the polymorphism may be performed by single strand conformation polymorphism analysis (SSCP). In this technique, PCR products from the region to be tested are heat denatured and rapidly cooled to avoid the reassociation of complementary strands. The single strands then form sequence dependent conformations that influence gel mobility. The different mobilities can then be analysed by gel electrophoresis.

Assessment may be by heteroduplex analysis. In this analysis, the DNA sequence to be tested is amplified, denatured and renatured to itself or to known wild-type DNA. Heteroduplexes between different alleles contain DNA “bubbles” at mismatched basepairs that can affect mobility through a gel. Therefore, the mobility on a gel indicates the presence of sequence alterations.

Restriction Site Based Methods

Where an SNP creates or abolishes a restriction site, the assessment may be made using RFLP analysis. In this analysis, the DNA is mixed with the relevant restriction enzyme (i.e. the enzyme whose restriction site is created or abolished). The resultant DNA is resolved by gel electrophoresis to distinguish between DNA samples having the restriction site, which will be cut at that site, and DNA without that restriction site, which will not be cut.

Where the SNP does not create or abolish a restriction site the SNP may be assessed in the following way. A mutant PCR primer may be designed which introduces a mutation into the amplification product, such that a restriction site is created when one of the polymorphic variants is present but not when another polymorphic variant is present. After PCR amplification using this primer (and another suitable primer or primers), the amplification product is admixed with the relevant restriction enzyme and the resultant DNA analysed by gel electrophoresis to test for digestion.

Thus if marker-specific restrictions sites do not pre-exist in the isolate to be assessed it may be created by modifying the sequence of cDNA or of the PCR-amplified segment by making appropriate changes in at least one oligonucleotide used for cDNA synthesis or for PCR.

Antibodies and Antibody Based Methods

The present invention also provides antibodies specific for the markers disclosed herein, in particular those which are capable of distinguishing the different forms of the marker (e.g. in EHV-1, the D₇₅₂N substitution in the ORF 30 DNA Pol gene). Methods of producing antibodies include immunising a mammal (e.g. human, mouse, rat, rabbit, horse, goat, sheep or monkey) with a polypeptide corresponding to the marker region. Antibodies may be obtained from immunised animals using any of a variety of techniques known in the art, and might be screened, preferably using binding of antibody to antigen of interest. The antibodies can then tested using conventional techniques for their ability to bind the other form of the marker, and those which are selective (capable of distinguishing the two, either through absolute binding or altered affinity) can be selected.

The use of such antibodies in methods described herein, e.g. for typing or assessing virulence, are also provided by the present invention. For instance, Western blotting techniques or immunoprecipitation may be used (Armitage et al, 1992, Nature 357: 80-82).

Antibodies may be polyclonal or monoclonal, and may optionally be modified in a number of ways. Indeed the term “antibody” should be construed as covering any specific binding substance having a binding domain with the required specificity. Thus, this term covers antibody fragments, derivatives, functional equivalents and homologues of antibodies, including any polypeptide comprising an immunoglobulin binding domain, whether natural or synthetic. Chimaeric molecules comprising an immunoglobulin binding domain, or equivalent, fused to another polypeptide are therefore included. Cloning and expression of Chimaeric antibodies are described in EP-A-0120694 and EP-A-0125023.

The invention will now be further described with reference to the following non-limiting Figures, Tables, Sequences and Examples. Other embodiments of the invention will occur to those skilled in the art in the light of these.

Legends

FIG. 1. Multi-locus sequence analysis.

Sequence analysis of a panel of isolates (originating from 63 separate outbreaks) for multiple loci is shown. For each ORF region, the results of sequence analysis are indicated by numbers according to the codes specified in Table 5 below. Each outbreak is coded according to the country of origin (GB=United Kingdom, US=U.S.A., PL=Poland), year of outbreak, identifier for that outbreak for the year and nature of disease, in the form ‘CountryYr/identifier(1,2,3 etc.)/disease(0=attenuated, 1=non-paralytic, 2=paralytic)’. For example, GB83/1/1 and GB83/2/1 indicate two independent outbreaks in the United Kingdom in 1983, without paralytic disease. Dashes (-) indicate the relevant sequence was not determined. * indicates the sequence data is incomplete for the given region, but the available data indicates the given code(s). Within each group, outbreaks have been sub-divided according to disease severity. Specific isolates mentioned in the text correspond to the following outbreaks: AB4-GB80/1/2, V592 - GB85/1/1, Army183 - US41/1/2, RacH-PL68/1/0, Rhinoquin-US72/1/0. RacH and Rhinoquin are vaccine strains that have been attenuated by multiple passage in tissue culture.

Table 1. Sequence Heterogeneity Between Individual Sequence Templates of V592

During analysis of the V592 sequence data a number of positions were noted where there were differences between the sequences determined from individual sequence templates, indicating that the V592 virion DNA population was heterogeneous at these positions. Each of the regions is listed (numbered according to the V592 genomic sequence, SEQ. ANNEX 1a) and details of the variation noted. NA indicates that the position lies outside of a recognised protein coding sequence (open reading frame: ORF).

Table 2. ORF Coding Changes Between V592 and AB4

V592 ORFs which have an altered amino acid sequence compared to the corresponding AB4 ORF are listed. The position of each change is numbered according to the V592 ORF amino acid sequences (SEQ. ANNEX 1b). For each position, the amino acid sequences for AB4 and V592 are shown. * indicates that the corresponding sequence is absent in the given strain—i.e. the other strain carries additional amino acids, usually due to variation in copy number of a nucleotide repeat element.

Table 3. Distribution of ORF30-ml Marker (G/A₂₂₅₄) Amongst Isolates From Paralytic or Non-Paralytic Outbreaks.

Isolates from a total of 59 different outbreaks (23 paralytic, 36 non-paralytic) were analysed to determine the sequence of the ORF30-ml marker (nucleotide position 2254). The table displays the number of occurrences of either G or A at position 2254 for each class of outbreak. The percentage of outbreaks for each class represented by either G or A is shown in italics. Data were analysed via the Fisher exact test to determine the significance of association of A₂₂₅₄ with non-paralytic outbreaks and G₂₂₅₄ with paralytic outbreaks, testing the null hypothesis that each marker is randomly distributed between the different classes of outbreak.

Table 4. Alignment of EHV-1 ORF30-ml Region With Other Herpesvirus DNA Polymerase Sequences

Various herpesvirus DNA polymerase sequences were aligned using CLUSTALW (using services at ANGIS, Australia) and the alignment surrounding the EHV-1 ORF30-ml (D/N₇₅₂) position is shown. Gaps are indicated by dashes (—). Viruses have been grouped as alpha-, beta- and gammaherpesviruses, or unclassified. In each case the abbreviated virus name and sequence accession number are shown (apart from EHV-1 strain V592, reported in this study (SEQ. ANNEX 3b)). The first position of each sequence is numbered; where the complete DNA polymerase sequence is not available, numbering of the partial sequence is shown in italics. The position of the D residue conserved in the majority of viruses, corresponding to the ORF30-ml (aa752) marker position, is shaded.

Table 5. Variable Sequence Marker Codes

Regions of variable sequence identified during multi locus sequence typing (see FIG. 1) are shown. For each region, the prototype (AB4) nucleotide sequence is shown for the relevant ORF and each of the sequence variants detected listed below in the form of an alignment, with the relevant code for each sequence (as used in FIG. 1) specified. Gaps in the sequence, due to nucleotide deletions compared with one or more of the other sequences, are indicated by dashes (—). For each sequence variant, positions identical to the AB4 sequence are shown by dots (.) and nucleotide differences specified. Sequences have been numbered according to the AB4 nucleotide sequence for each of the ORFs, Blocks of sequence which are non-contiguous are indicated by intervening echelons (\hat{ }). For ORF37, the region of sequence displayed extends beyond the ORF stop codon. Nucleotides after the stop codon are shown in lowercase and are numbered in italics.

SEQ. ANNEX 1. a) Complete Genomic Sequence of EHV-1 Strain V592; b) Feature Table

Sequence data was compiled using a combination of random shotgun sequencing and targeted sequencing to fill gaps. Where there was sequence heterogeneity between individual sequencing templates (as listed in Table 1), the majority consensus sequence is shown. The top strand of the sequence is displayed (1a), according to the standard orientation of the genome adopted for EHV-1 strain AB4 [19]. The feature table (1b) was compiled using the Sequin programme (National Center for Biotechnology Information, NIH, U.S.A.) and displays features relating to the recognised open reading frames (ORFs) and nucleotide repeat regions, numbered according to the complete genomic sequence (1a). Features listed are in the orientation of the top strand of the sequence, unless specified as ‘complement’ (bottom strand, reverse direction).

SEQ. ANNEX 2. PCR Amplification and Sequencing Primers

Oligonucleotide primers used for amplification and sequencing of selected ORF regions, including regions of sequence variation between AB4 and V592, are shown.

SEQ. ANNEX 3. a) DNA Sequence of EHV-1 (Strain V592) ORF30; b) Amino Acid Sequence of EHV-1 (Strain V592) ORF30

For each sequence, residues altered for strain V592 compared with strain AB4 are underlined.

### EXAMPLES

### Example 1

### Determination of the Complete Genomic Sequence of EHV-1 Strain V592

We determined the V592 genomic sequence (149,430 bp) via shotgun cloning and sequencing of viral DNA prepared from purified virions. Virus supernatant was prepared from equine embryonic lung cells infected at low multiplicity (<0.001 pfu/ml), virions purified by sucrose gradient sedimentation and DNA extracted from purified virions essentially as described by Telford et al [19]. Viral DNA was self ligated, sonicated to generate random fragments and, following end repair, cloned into M13mp19. In addition, a panel of ‘semi-random’ clones was generated by digestion of viral DNA with frequent cutting (blunt ended) restriction enzymes (AluI, PvuII, BalI), followed by cloning into M13mp19. In the final stages of sequencing, specific regions spanning sequence data ‘gaps’ were amplified by PCR, blunt ended and cloned into M13mp19. Single stranded M13 templates were prepared and sequenced using proprietory sequencing reagents, and samples analysed on either an ABI 377 or ABI 9600 automated sequencer. Sequence reads were assembled using the Staden sequence analysis programs PREGAP4 and GAP4 [21]. The complete genomic sequence of EHV-1 strain V592 is shown in SEQ. ANNEX 1. It should be noted that the attached sequence is the consensus for the majority of sequence reads. The V592 stock used to generate DNA for sequencing has not been plaque purified and consequently contains a mixture of DNA populations at certain sites. Sites showing heterogeneity between individual sequencing templates are shown in Table 1. These sites are all regions of variable repeat length apart from one position of single nucleotide heterogeneity.

### Example 2

### Comparison Between the Genomic Sequences of V592 and AB4

The AB4 complete sequence was included in the V592 sequence assembly to enable positions of variation between the two sequences to be identified using GAP4. Positions of variation were then analysed using the OMIGA software (Oxford Molecular Ltd.) to determine changes resulting in amino acid coding changes for known ORFs. Table 2 shows all of the coding changes identified. A total of 31 out of 76 ORFs were found which possess amino acid variation between the two strains. Two ORFs (24 and 71) showed variable copy numbers of nucleotide repeat elements. Previous studies had indicated variation in repeat element copy number for ORF 24 and ORF71 among EHV-1 field isolates [16, 17]. Such regions are inherently unstable and therefore of limited use for epidemiological studies. ORF 14 of V592 carried a 9 bp duplication, resulting in insertion of 3 amino acids. ORF 68 displayed the most significant change, with a single nucleotide deletion in V592 (8 Gs in AB4, 7Gs in V592) resulting in a frameshift and hence multiple coding changes and premature truncation. Such a frameshift had previously been noted for several other EHV-1 isolates [22]. The other variable ORFs possessed minor changes between AB4 and V592, usually comprising a single amino acid substitution.

### Example 3

### Evaluation of Genetic Heterogeneity of EHV-1 Field Isolates

As noted above, EHV-1 infection results in outbreaks of varying severity, including neurological (paralytic) disease, induction of abortion or mild respiratory symptoms. Previous evidence had indicated genetic variability of EHV-1 field isolates but had not demonstrated conclusively the presence of distinct ‘strains’ of EHV-1. Having identified genetic differences between a ‘paralytic’ (AB4 and non-paralytic (V592) strain of EHV-1, we sought to test the following hypotheses:
1) Positions of sequence variation between AB4 and V592 are indicative of regions of sequence variability amongst EHV-1 field isolates and will therefore provide markers enabling discrimination between EHV-1 strains.
2) AB4 is representative of a distinct group of EHV-1 strains capable of causing paralytic disease.
3) One or more specific sequence ‘markers’ which vary between AB4 and V592 are indicative of strains capable of causing paralytic disease.

In order to test the above hypotheses, we assembled DNA samples prepared from a panel of field isolates (from the U.K. and North America) recovered from outbreaks of varying disease severity, collected over the course of 30 years. A subset of the ORFs with observed coding changes were selected for preliminary analysis, namely ORFs 8, 11, 14, 15, 30, 33, 34, 37, 39, 40, 52, 67, 68, 73. For each of these ORFs, PCR primers were designed for amplification of regions of sequence variability and for sequencing, as listed in SEQ. ANNEX 2. PCR products were purified and sequenced (ABI 9600) and the results assembled using the DNASTAR software package. For each ORF region analysed, positions of variable sequence were noted as listed in Table 5.

### Example 4

### Multi-Locus Sequence Typing Discriminates Between EHV-1 Strains Circulating in the Field

The results obtained from multi-locus sequence typing of the panel of field isolates provide support for Hypothesis 1, namely that the loci of variable sequence noted in the comparison of the AB4 and V592 genomes are suitable as markers for discriminating between strains of EHV-1 circulating in the field. In particular, the ORF68 region sequencing results are particularly useful, since this locus displays an unusually high frequency of variable nucleotides and grouping of isolates according to their ORF68 sequences correlates well with other variable loci tested, as summarised in

FIG. 1. Where multiple isolates from a given outbreak have been characterised, they have provided consistent results for the markers tested. Significantly, it appears that six major distinct groups of related strains are identifiable (on the basis of ORF68 sequences and supported by other markers tested), isolated from outbreaks that have occurred over the course of 20-30 years. The characteristics of these groups are summarized below:
Group 1 Includes AB4 (from outbreak GB80/1/2) and isolated from an additional four U.K. outbreaks, occurring between 1980 and 1993. Three outbreaks of abortion without paralysis, one of paralysis and abortion. An isolate from a single U.S.A. outbreak (abortion without paralysis, 1985) is also placed in this group, since it carries an ORF6B which is frameshifted compared to the predominant form (although this has 9 rather than 8 Gs at the ORF68 frameshift position and is therefore distinct from other members of group 1).
Group 2 Isolates from eleven U.S.A. outbreaks and six U.K. outbreaks, occurring between 1970-2003. Nine outbreaks of abortion without paralysis, eight of paralysis (with or without abortion). This group is closely related to Group 1 (but with 7 rather than 8 Gs at the ORF68 frameshift position).
Group 3 Isolates from seventeen U.K. and one U.S.A outbreaks, occurring between 1981-2003. One respiratory only, fourteen outbreaks of abortion without paralysis, three of paralysis (with or without abortion).
Group 4 solates from five U.K. and one U.S. outbreaks, occurring between 1980-2000. One respiratory only, two outbreaks of abortion without paralysis and three of paralysis (with or without abortion).
Group 5 Isolates from nine U.S.A. outbreaks, occurring between 1975 -2002. Two of abortion without paralysis and seven of paralysis (with or without abortion). This group also contains Army 183 (US41/1/1/2: an experimental strain originally isolated in the U.S.A. which causes abortion and paralysis), RacH (PL68/1/0: a tissue culture passaged, attenuated vaccine strain originally isolated in Poland), and Rhinoquin (US72/1/0: a tissue culture passaged, attenuated vaccine strain originally isolated in the U.S.A.)
Group 6 Includes V592 (GB85/1/1) and isolates from three other U.K. outbreaks, occurring between 1985-2001. One respiratory outbreak, three of abortion without paralysis.

The above group assignments were made on the basis of the ORF68 marker. These assignments are supported by the following general rules applying to other markers (although it should be noted that not all of the markers have been determined for all of the isolates within each group).

ORF14 Majority of isolates with type 2A sequence in groups 3 and 4 ORF33 Majority of isolates with type 1 sequence (both 33-ml and 33-m2) in groups 1 and 2 ORF37 Type 1A sequence predominantly in groups 1 and 2 ORF39 Majority of isolates with type 2 sequence in groups 3, 4 or 5 ORF52 Type 1 sequence predominantly in groups 1 and 2

From consideration of the above, Groups 1 and 2 are related to each other and similarly Groups 3 and 4 are related. Group 6 possesses characteristic unusual sequence for ORFs 8, 11, 30-m2, 34, 39, 40, 67 and 73.

The observation of genetically related strains being recovered from outbreaks separated by many years support the proposal that distinct strains of EHV-1 circulate in the field and that such strains are relatively genetically stable over time. Furthermore, there is evidence for geographical restriction in strain circulation, with certain of the groups consisting of predominantly U.K. (Groups 1, 3, 4 and 6) or U.S.A. (Group 5) isolates.

### Example 5

### Distinct Strain Groups of EHV-1 Do Not Appear to be Associated With Outbreaks of Severe (Paralytic) Disease

The data disprove Hypothesis 2, since it is clear that isolates genetically distinct from AB4 have been recovered from outbreaks of paralytic disease. Furthermore, the isolate groupings as described above do not appear to segregate clearly according to outbreak severity, since isolates from paralytic outbreaks are found in five of the six groups. Group 6 does not contain any isolates from paralytic outbreaks and may represent isolates with reduced neurovirulence, but more data are required to test this possibility (currently only four outbreaks are represented in Group 6). Similarly, the majority (15/18) of isolates in Group 3 are from non-paralytic outbreaks, suggesting that members of this group may have reduced neurovirulence. Conversely, the majority of isolates in Group 5 (8/10) are from paralytic outbreaks. However, consideration of ORF30 sequence variation (see Example 6 below) suggests strongly that this particular marker, rather than the strain grouping, correlates with disease severity.

### Example 6

### Sequence Variation in ORF30 (DNA Polymerase) Correlates Strongly With Disease Severity

As mentioned above, most of the markers tested show good correlation with the strain groupings identified according to the ORF68 sequences.

Notably, this is not the case for one of the variable sequences within ORF30. The complete nucleotide and amino acid sequence of EHV-1 strain V592 ORF30 is shown in SEQ. ANNEX 3. Three nucleotide changes are present compared with the AB4 ORF30 sequence (accession no. AAB02465), namely C₉₂₄-T (non-coding change), G₂₂₅₄-A (amino acid change D₇₅₂-N) and G₂₉₆₈-A (amino acid change Eggo-K)

Of these three positions, the G/A₂₂₅₄ polymorphism was found to show a very strong correlation with isolates from outbreaks of paralytic disease, with the majority of ‘paralytic’ isolates (83%) having G₂₂₅₄ whereas 100% of the non-paralytic isolates had A₂₂₅₄ (Table 3. This includes data from all the outbreaks listed in FIG. 1, with the exception of the attenuated vaccine strains RacH (PL68/1/0) and Rhinoquin (US72/1/0)). This association was highly significant (p<0.0001). It should also be noted that three of the four ‘paralytic’ isolates with A₂₂₅₄ were from single cases of paralytic disease, rather than multi-case outbreaks.

In addition to the isolates from field outbreaks, two tissue culture adapted attenuated vaccine strains of EHV-1 were analysed. Both of these vaccine strains were found to carry the G₂₂₅₄ paralytic' marker. Isolate T501 (designated as US72/1/0) is a prototype, attenuated vaccine strain (‘Rhinoquin’) which had been developed and tested in the U.S.A. [23]. Although found to be attenuated in preliminary laboratory studies, this virus was associated with abortions and paralytic disease when studied (as a live vaccine) in large scale field trials; as a consequence, development of the Rhinoquin vaccine ceased [24]. The second attenuated vaccine strain (RacH, designated PL68/1/0 (25]) , in contrast to Rhinoquin, has a good safety record. It may be significant, therefore, that RacH carries a second variant nucleotide close to G₂₂₅₄, namely C₂₂₅₈. This sequence is not present in any other of the isolates tested, and results in a coding change Y₇₅₃-S.

A second single nucleotide polymorphism, A₂₂₇₉-G (marker ORF30-ml; code 2B) was noted which was present in two paralytic isolates which had A₂₂₅₄ rather than G₂₂₅₄ for ORF30. One of these isolates (T937, outbreak US99/3/2) is from a single case of paralytic disease, while the other (T949, outbreak US02/1/2) is from a multi-case outbreak of paralytic disease. Accordingly, this infrequent ORF30 sequence variant may also predispose to paralytic disease.

Interestingly, another ORF30 coding change variant (marker ORF30-m2: G/A₂₉₆₈) did not correlate with disease severity. Of the isolates tested, the majority had G₂₉₆₈, including representatives from paralytic and non-paralytic outbreaks. All of the isolates with A₂₉₆₈ were within Group 6, i.e. V592 related (see FIG. 1).

### Example 7

### Comparison Between EHV-1 ORF30 Sequence Variation and Other Herpesviruses

The observed sequence variation for EHV-1 at ORF30 amino acid position 752 occurs at a conserved position of the herpesvirus DNA polymerase. An alignment of the selected region for certain alpha, beta, gamma and unclassified herpesviruses is shown in Table 4. The sequence of strain AB4 (D₇₅₂) conforms to all of the herpesviruses DNA pol sequences shown in Table 4, with the exception of PRV, which has a conserved amino acid substitution (E) at this position. The sequence observed for V592 and the majority of EHV-1 field isolates (N₇₅₂) is therefore not conserved with the vast majority of other herpesviruses, including those of mammals, birds, reptiles, amphibians and fish.

This raises the interesting possibility that the progenitor EHV-1 virus is likely to have encoded D at position 752 of DNA pol. Subsequently, mutation at this position to N₇₅₂ may have occurred and the resulting strains may have had a selective advantage (eg. due to more efficient establishment of latency/reactivation or improved transmission, possibly via reduced virulence and hence increased carriage long-term within horse populations) and hence now predominate. If so, the D₇₅₂ marker characteristic of paralytic strains results from back-mutation to the progenitor sequence. The adjacent residue (753) is also conserved as tyrosine or other hydrophobic residues. It may be significant, therefore, that RacH (which carries the D₇₅₂ marker but has a good safety record in the field) has a non-conservative mutation at this position (S₇₅₃), which may have possibly prevented recombination around this region with naturally occurring isolates resulting in generation of viruses with paralytic potential.

As described in Example 6, two paralytic isolates were identified which are unusual in encoding N (rather than D) at position 752, and which carry a novel nucleotide substitution (A₂₂₇₉-G) resulting in an amino acid change from D-G at position 760. It is interesting to note from the alignment (Table 4) that the consensus amino acid at the equivalent position for alphaherpesviruses is G, with only EHV-1 (AB4 and V592) and EHV-4 ORF30 found to encode D at this position. Thus, the D₇₆₀-G variation is analogous to the N₇₅₂-D variation, i.e. a change resulting in the amino acid conforming to the consensus at this position (for at least the alphaherpesviruses) is found in paralytic EHV-1 isolates. Thus the D₇₆₀-G amino acid change may represent an atypical EHV-1 ORF30 mutation which results in a virus with increased potential for paralytic disease.

Material and Methods

Reagents

PCR reagents were obtained from Applied Biosystems International (USA), Genset (France) and Invitrogen (USA). Restriction enzymes were obtained from Applied Biosystems (USA) and Promega (USA). An Amicon Microcon Filter YM100 kit (Millipore, USA) was used to purify PCR products and DNA quantitation standards were obtained from Whatman Bioscience (UK). Sequencing reagents were obtained from Applied Biosystems International (USA) and Web Scientific (UK).

Viruses and Cells

Virus field isolates were obtained from the archive material held at the Animal Health Trust and Gluck Equine Research Institute. Where necessary, virus isolates were propagated in equine derived cell lines (fibroblast) and infected cells used for the preparation of viral DNA.

Preparation of Viral DNA

i) Large Scale, Virion DNA (Strain V592) and ‘Shotgun’ Cloning

DNA was prepared from purified virions essentially as described by Dumas et al 1980, Telford et al, 1992 and Rawlinson et al 1996. Equine fibroblasts (embryonic lung cells) were infected at low multiplicity and incubated at 37° C. until complete cytopathic effect (c.p.e.) had developed. The tissue culture medium was harvested, centrifuged at low speed (1500g, 5 mins) to remove cell debris and the supernatant centrifuged at high speed (17,000g, 200 mins) to pellet virions. Pellets resuspended in 5ml MEM/2%FCS and then purified via a sucrose gradient (40%/60% sucrose in PBS), with high speed centrifugation (69,000 g, 120 mins). Following collection of the virion material present at the sucrose gradient interface, the virus was pelleted by centrifugation (17,000 g, 180 mins) and the pellet resupended in 1 ml TE buffer (10 mM Tris, 0.1 mM EDTA, pH 8). Virion DNA was prepared by Proteinase K/SDS digestion followed by phenol/chloroform extraction and ethanol precipitation. The DNA (re-dissolved in TE buffer) was self ligated (T4 DNA ligase) to remove free ends and then sonicated using a cup horn device (Ultrasonic processor XL, MISONIX, U.S.A.) to generate randomly sheared fragments. Sonicated DNA was then gel purified (QIAEX kit, QIAGEN Ltd, U.K.), selecting fragments with an approximate size range of 300-1,000 bp. Fragments were cloned into either M13mp18 or M13mp19 via blunt end cloning, using the Novagen ‘Perfectly Blunt’ kit (CN Biosciences (UK) Ltd., U.K.). Additional blunt ended fragments were generated by restriction enzyme digestion using frequently cutting enzymes.
ii) Small scale, virus isolate DNA (various field isolates) Equine fibroblasts were infected (various multiplicities, depending on the isolate) and incubated (37° C.) until 50-100% c.p.e. had developed. Cells were then pelleted by centrifugation, washed with TE buffer and resuspended in Proteinase K/SDS solution 0.1 mg/ml Proteinase K, 0.5% SDS). Following 1-2 hours digestion, the DNA was prepared by phenol/chloroform extraction followed by ethanol precipitation. The purified DNA was re-dissolved in TE buffer.
   Preparation of Single Stranded M13 Sequencing Templates

Following ligation of randomly sonicated fragments or blunt ended restriction enzyme products derived from V592 virion DNA, ligation reactions were transformed into E. coli and plated onto agar (supplemented with X-gal) for selection of white plaques (according to manufactures instructions: Novagen ‘Perfectly Blunt’ kit). Individual plaques were picked into TE buffer before being used to infect E. coli cultures to prepare sequencing templates, in accordance with methods given in the ‘ABI Prism DNA Sequencing Guide’ (Applied Biosystems, USA).

PCR Amplification and Purification.

The PCR mix (50 μl) consisted of 0.3 μM of each primer (Genset), 0.2 mM of each NTP (Applied Biosystems), 3×PCRx Enhancer solution (Invitrogen) and 1.25 U/μl AmpliTaq DNA polymerase (Applied Biosystems) in 10 mM Tris-HCL (pH 8.3) solution containing 1.5 mM MgCL₂ (Applied Biosystems). The PCR reaction was denatured for 4 min at 94° C., then cycled for 32 cycles at 94° C. for 30 seconds, 1 min at the annealing temperature of the primers used, and 2 min at 72° C. followed by a final step of 10 min at 72° C. After cycling, 10 μl of each PCR product was size fractionated on a 0.7% agarose gel containing ethidium bromide. Following product identification, the PCR products were purified using an Amicon Microcon Filter YM100 kit and quantified by size fractionation, on a 2% agarose gel containing ethidium bromide, with DNA Quantification standards (Whatman Bioscience). The purified PCR products were either cloned into M13mp19 (gap filling for determination of V592 sequence) or directly sequenced using EHV-1 specific sequencing primers (SEQ. ANNEX 2).

Sequencing Reactions and Sequence Analysis

Templates (M13 or PCR products) were sequenced using ABI sequencing reagents (dRhodamine or Big Dye) according to manufacturer's instructions. Reaction products were analysed using either an ABI 377 or ABI 3600 automated sequencer. Trace files were downloaded and processed for assembly and analysis using either:
a) the Staden suite of programmes [21], in particular PREGAP4 and GAP4, run using either a LINUX or Windows NT platform. Further details of the Staden programmes may be found at: ‘www.mrc-lmb.cam.ac.uk/pubseq/2 .
b) the DNASTAR programme SeqManII (version 4.03, DNASTAR, Inc., U.S.A.))

Following sequence assembly, further analysis, in particular identification of coding changes and comparison between homologous genes between different herpesviruses, was carried out using the OMIGA software package (version 2.0, Oxford Molecular Ltd., U.K.)

References

1. Blunden, A. S., Whitwell, K. E. & Pegler, K. M. (1993). An outbreak of paralysis associated with equine herpes virus type 1 infection in a livery stable. Progress in Veterinary Neurology 3:95-100.
2. Crowhurst, F. A., Dickinson, G. & Burrows, R. (1981). An outbreak of paresis in mares and geldings associated with equid herpesvirus 1. The Veterinary Record 109:527-528.
3. Greenwood, R. E. S. & Simson, A. R. B. (1980). Clinical report of a paralytic syndrome affecting stallions, mares and foals on a thoroughbred studfarm. Equine Veterinary Journal B12B:113-117.
4. McCartan, C. G., Russell, M. M., Wood, J. L. N. & Mumford, J. A. (1995). Clinical, serological and virological characteristics of an outbreak of paresis and neonatal foal disease to equine herpesvirus-1 on a stud farm. The Veterinary Record 136: 7-12.
5. Mumford, J. A. & Edington, N. (1980). EHV-1 and equine paresis. The Veterinary Record 106:277.
6. Allen, G. P., Kydd, J. H., Slater, J. D. and Smith, K. C. (1999). Recent advances in understanding the pathogenesis, epidemiology and immunological control of equid herpesvirus-1 (EHV-1) abortion. In: Equine Infectious Diseases VIII (eds. U Wernery, J. F. Wade, J. A. Mumford and O-R Kaaden) R&W Publications (Newmarket) Limited. pp129-146.
7. Edington, N., Bridges, C. G. & Patel, J. R. (1986). Endothelial cell infection and thrombosis in paralysis caused by equid herpesvirus-1: equine stroke. Arch. Virol. 90:111-124.
8. Mumford, J. A., Hannant, D., Jessett, D. M., O'Neill, T., et al. (1994). Abortigenic & neurological disease caused by experimental infection with Equid herpesvirus-1. Equine Infectious Diseases VII (Proceedings), 261-275
9. Platt, H., Singh, H. and Whitwell, K. E. (1980). Pathological observations on an outbreak of paralysis in broodmares. Equine Veterinary Journal 12: 118-126.
10. Whitwell, K. E. & Blunden, A. S. (1992). Pathological findings in horses dying during an outbreak of the paralytic form of Equid herpesvirus type 1 (EHV-1) infection. Equine Veterinary Journal 24: 13-19.
11. Patel J R, Edington N, Mumford J. A. (1982). Variation in cellular tropism between isolates of equine herpesvirus-1 in foals. Arch. Viol. 74: 41-52.
12. Mumford, J A, Rossdale, P D, Jesset, D M, Gann, S., Ousey, J. and Cook, R F (1987). Serological and virological investigations of an equid herpesvirus-1 (EHV-1) abortion storm on a stud farm in 1985. Journal of reproduction and Fertility 35 (Suppl.): 509-518.
13. Smith, K. C., Whitwell, K. E., Mumford, J. A., Hannant, D., Blunden, A. S., and Tearle, J. P. (2000). Virulence of the V592 isolate of equid herpesvirus type 1 in ponies. J. Comp. Path. 122:288-297.
14. Allen, G. P., Yeargan, M. R., Turtinen, L. W., Bryans, J. T. and McCollum, W. H. (1983). Molecular epizooitologic studies of equine herpesvirus-1 infections by restriction endonuclease fingerprinting of viral DNA. Am. J. Vet. Res. 44, 263-271.
15. Allen, G. P., Yeargan, M. R., Turtinen, L. W. and Bryans, J. T. (1985). A new field isolate of equine abortion virus (equine herpesvirus-1 among Kentucky horses. Am. J. Vet. Res. 46:138-140
16. Binns, M., McCann, S., Zhang, L., Wood, J. & Mumford, J. A. (1994). Molecular epidemiology of EHV-1 and EHV-4: a search for variable restriction sites. Equine Infectious Diseases VII (Proceedings), 237-241.
17. McCann, S. H. E., Mumford, J. A. & Binns, M. M. (1995). Development of PCR assays to detect genetic variation amongst equine herpesvirus-1 isolates as an aid to epidemiological investigation. Journal of Virological Methods 52: 183-194.
18. Studdert, M. J, Crabb, B. S. and Ficorilli, N. (1992). The molecular epidemiology of equine herpesvirus 1 (equine abortion virus) in Australasia 1975-1989. Australian Veterinary Journal 69:104-111.
19. Telford, E. A. R., Watson, M. S., McBride, K. & Davison, A. J. (1992). The DNA sequence of equine herpesvirus-1. Virology 189:304-316.
20. Telford, E. A. R., Watson, M. S., Perry, J, Cullinane, A. A. and Davison, A. J. (1998). DNA sequence of equine herpesvirus-4. J Gen Virol 79: 1197-1203.
21. Staden, R. (1996). The Staden Sequence Analysis Package. Molecular Biotechnology 5: 233-241.
22. Meindl, A., Osterrieder, N. (1999). The equine herpes 1 Us2 homolog encodes a non-essential membrane-associated virion component. J. Virol 73:3430-3437.
23. Purdy C W, Ford S. J. and Grant W. F. (1977). Equine rhinopneumonitis virus (herpesvirus type 1): attenuation in stable monkey cell line. Am J Vet Res. 38:1211-1215.
24. Kit M and Kit S. U.S. Pat. No. 05,292,653.
25. Meyer H, Hubert P H, Eichhorn W. (1987) Changes in restriction enzyme pattern of the equine herpes virus type 1 (EHV-1) strain Rac H DNA during attenuation. J. Vet. Med[B]. 34:310-3.
26. Wilson (1997). Equine herpesvirus 1 myeloencephalopathy. Vet Clin North Am Equine Pract. 13: 53-72
27. Wang, T. S.-F. (1991). Eukaryotic DNA polymerases. Ann. Rev. Biochem. 60:513-552.
28. Hwang, C. B., Ruffner, K. L. and Coen, D. M. (1992). A point mutation within a distinct conserved region of the herpes simplex virus DNA polymerase gene confers drug resistance. J. Virol. 66:1774-1776.
29. Chou, S., Lurain, N., Weinberg, A., Cai, G-Y, Sharma, P., Crumpacker, C. S., Adult AIDS Clinical Trials Group CMV Laboratories. (1999). Antimicrob. Agents and Chemother. 43: 1500-1502.
30. Matthews, J. T., Terry, B. J. and Field, K. A. (1993). The structure and function of the HSV DNA replication proteins: defining novel antiviral targets. Antiviral; Res. 20: 89-114.

31. Kamiyama, T., Kurokawa, M. and Shiraki, K. (2001). Characterisation of the DNA polymerase gene of varicella-zoster viruses resistant to acyclovir. J. Gen. Virol. 82: 2761-2765.

TABLE 1
Sequence heterogeneity between individual sequence
templates of V592

| Position¹ | ORF | Variation |
|---|---|---|
| 105-308 | NA | 10-13 copies 17 bp repeat element |
| 366-707 | NA | 14-19 copies 18 bp repeat element |
| 1959 | 2 | substitution A-G in minority of reads (2/9) |
| 73803-74090 | NA | 8-9 copies 32 bp repeat element |
| 108895-108984 | NA | 4-5 copies 18 bp repeat element |
| 112316-112419 | NA | 8-9 copies 13 bp repeat element |
| 123549-123656 | NA | 2-4 copies 27 bp repeat element |
| (138661-138768) | | |
| 129328-129552 | 71 | 17-18 copies 15 bp repeat element |

¹Numbered according to V592 genome

TABLE 2
ORF coding changes between V592 and AB4

| ORF | Position¹ | AB4 sequence | V592 sequence |
|---|---|---|---|
| 2 | 59 | G | D |
| 5 | 114 | G | V |
| 8 | 114 | D | N |
| 11 | 189 | Q | K |
| 13 | 305 | S | L |
| | 460 | A | T |
| 14 | 619-621 | * | PSR |
| | | | 9 bp duplication resulting in 3 amino acid insertion |
| 15 | 166 | D | N |
| 22 | 430 | S | P |
| 24 | 2567-2568 | (PTLPPAPPLPQSTSKAASGPP)₂ | * |
| | | | 126 bp deletion (2 copies 63 bp repeat element) |
| | | | resulting in 42 amino acid deletion |
| | 2586 | G | S |
| | 2829-2836 | * | AKDQAKDQ |
| | | | 24 bp insertion (2 copies 12 bp repeat element) |
| | | | resulting in 8 amino acid insertion |
| | 2904 | E | K |
| | 2913-2927 | * | PTGAVPENTPLPDDS |
| | | | 45 bp insertion resulting in 15 amino acid insertion |
| | 3099 | T | A |
| 29 | 12 | T | K |
| 30 | 752 | D | N |
| | 990 | E | K |
| 31 | 90 | N | S |
| 32 | 42 | S | L |
| 33 | 15 | N | H |
| | 976 | N | D |
| 34 | 66 | D | G |
| 36 | 47 | S | R |
| 37 | 265 | A | V |
| 39 | 440 | S | L |
| 40 | 196 | R | H |
| 42 | 1275 | K | R |
| 45 | 427 | E | G |
| 46 | 140 | F | S |
| 50 | 367 | P | S |
| 52 | 386 | A | V |
| 57 | 804 | K | R |
| 64 | 73 | T | A |
| | 648 | T | S |
| 67 | 261 | S | F |
| 68 | 210 | R | H |
| | 247 | D | M - due to single nucleotide (C) deletion resulting |
| | | | in frameshift relative to AB4. All downstream |
| | | | sequence divergent from AB4. Resulting polypeptide |
| | | | 303 amino acids long (cf. 418 for AB4) |
| 71 | 226-227 | SS | TA |
| | 231-299 | * | TAATTTAATTSSATTAATTSS(TTTAA)₉TTT |
| | | | additional copies 15 bp repeat elements and |
| | | | nucleotide substitutions, resulting in 69 amino |
| | | | acid insertion². |
| 73 | 122 | A | V |
| 76 | 128 | F | S |

¹Numbered according to the V592 amino acid sequence.
²Variable copy number in V592 - majority sequence shown

TABLE 3
Distribution of ORF 30-ml marker amongst outbreaks of
paralytic or non-paralytic disease in the U.K. and U.S.A.

| Type of outbreak | G₂₂₅₄ | A₂₂₅₄ | Total |
|---|---|---|---|
| Non-paralytic | 0 | 36 | 36 |
| Percentage | 0% | 100% | |
| Paralytic | 19 | 4 | 23 |
| Percentage | 83% | 17% | |
| Total | 19 | 40 | 59 |

Significance of association: p < 0.0001 (Fisher exact test)

[image] TABLE 5
Variable Sequence Marker Codes

ORF8
CODE 331
1 CGTCTCTCGG
2 .........A
ORF11
CODE 560
1 AGAGTCAGTG
2 .....A....
ORF14
CODE 1841 1851 1861 1871 1881 1891 1901
1 TGCGCCCCAG C---------CGTGCCGGG CGTTCCCGCG TGAGAGTGGA AGACCAAACT CTGGAACCAT CGTCCCCCGC
1A .......... .---------......... .......... ...A...... .......... .......... ..........
1B .......... .---------......... .......... ...C...... .......... .......... ..........
2 .......... .CGCCCCAGC......... .......... .......... .......... .......... ..........
2A .......... .CGCCCCAGC......... .......... ...A...... .......... .......... ..........
2B .......... .CGCCCCAGC......... .......... .......... .......... .......... ........A.
3 .......-.. .CGCCCCAGC......... .......... .......... .......... .......... ..........
ORF15 490
1 AACCTCGATG
2 ......A...
ORF30-m1
CODE 2251 2261 2271 2281 2291 2301
1 GTCGACTACT CGACGTTCGA GGTGGGTGAC CAAAAGTTAT TTTTTGTCCA CGCCCATATT
1A .......C.. .......... .......... .......... .......... ..........
2 ...A...... .......... .......... .......... .......... ..........
2A ...A...... .......... .......... .......... .......... ....A.....
2B ...A...... .......... ........G. .......... .......... ..........
ORF30-m2
CODE 2961
1 GGCAGCAGAG
2 .......A..
ORF33-m1
CODE 41
1 GCAATTGGCG
2 ..C.......
ORF33-m2
CODE 2921
1 TGGAAAATGA
2 .....G....
ORF34
CODE 151 \hat{ } 191 \hat{ } 301
1 CAACAGACAA \hat{ } CGGACGATCT \hat{ } GCCTGCCGGG
2 .....T.... \hat{ } .......... \hat{ } ..........
2A .....T.... \hat{ } .......... \hat{ } ..A.......
2B .....T.... \hat{ } .......... \hat{ } ......T...
3 .......... \hat{ } ......G... \hat{ } ..........
0RF37
CODE 791 801 811 821 831 841 851
1 GGGCGGCGTC CCTTTTTTCC CCAAAATAAa agccggtgca attaaagacg agtgcccctt tttt-gtcggc
1A .......... .......... .......... .......... .......... .......... ....t......
2 ...T...... .......... .......... .......... .......... .......... ....-......
ORF39
CODE 1311 \hat{ } 1561
1 AATAGTGTCA \hat{ } CCCGAGCCAG
2 .......... \hat{ } ........C.
3 ........T. \hat{ } ........C.
ORF40
CODE 491 \hat{ } 581
1 TCTACACCCC \hat{ } TTGATCGTAT
2 .......T.. \hat{ } ......A...
ORF52
CODE 1151
1 GATACGCCAA
2 .......T...
ORF67
CODE 751 781
1 GCCAGGCAGC \hat{ } TCTGCAGAAA
1A ........A. \hat{ } .T........
2 .......... \hat{ } .T........
ORF68
CODE 331 341 \hat{ } 621 / 701 711 721 731 741 751
1 CATCTCAACT CCAGCCTTAT \hat{ } ATTAGTTCGT \hat{ } GGCGGCCGCT GCCGCGGCGG CGGCCGTCGG AGGGGGGGG-A TGCGCGCCCC
GAGGCGGCGC
1A .......... .......... \hat{ } .......... \hat{ } .......... .......... .......... .........G. ..........
..........
2 .......... .......... \hat{ } .......... \hat{ } .......... .......... .......... ........-.. ..........
2A .......... .......... \hat{ } .......... \hat{ } .........C .......... .......... ........-.. ..........
..........
3 .......... .......... \hat{ } ........A. \hat{ } .......... ........T. .......... ........-.. ..........
..........
3A .......... .......... \hat{ } ........A. \hat{ } .........A ........T. .......... ........-.. ..........
..........
4 .......... .......... \hat{ } ........A. \hat{ } .......... .......... .......... ........-.. ..........
..........
4A .......... ...A...... \hat{ } ........A. \hat{ } .......... .......... .......... .......-.. ..........
..........
4B .......... .......... \hat{ } ........A. \hat{ } .......... .......... .......... .......-.. ..........
....T.....
5 .......... .......... \hat{ } ........A. \hat{ } .........G ..A....... .......... .......-.. ..........
..........
6 .....T.... .......... \hat{ } ........A. \hat{ } .......... .......... .......... .......-.. ..........
....T.....
ORF73
CODE 360
1 CAATGCCTCT
2 .....T....

1. A method for sub-typing, classifying, identifying or monitoring an Equine herpesvirus type 1 (EHV-1) isolate, the method comprising use of a genetic marker shown in Table 5.

2. A method as claimed in claim 1 for sub-typing the EHV-1 isolate into one of the 6 groups shown in FIG. 1.

3. A method as claimed in claim 2 wherein the marker is an ORF68 polymorphic sequence shown in Table 5.

4. A method for assessing the virulence of an Equine herpesvirus type 1 (EHV-1) or type 4 (EHV-4) isolate, the method comprising use of a genetic marker.

5. A method as claimed in claim 4 wherein the marker is a DNA polymerase (ORF30) marker.

6. A method for assessing the virulence of a herpesvirus isolate, the method comprising use of virulence marker corresponding to an DNA polymerase ORF30-ml region virulence marker.

7. A method as claimed in claim 6 wherein the herpesvirus isolate is an Equine herpesvirus type 1 (EHV-1) or type 4 (EHV-4) isolate.

8. A method as claimed in claim 6 wherein the marker is a polymorphic marker.

9. A method as claimed in claim 6 which is used to assess neurovirulence.

10. A method as claimed in claim 6 wherein a marker corresponding to ORF30 amino acid position 752 and\or 760 of the EHV-1 V592 is assessed.

11. A method as claimed in claim 10 wherein the presence of an acidic amino acid at the position corresponding to 752 and\or a non-acidic amino acid at the position corresponding to 760 is correlated with higher virulence of the herpesvirus.

12. A method as claimed in claim 11 wherein the acidic amino acid is Asp and non-acidic amino acid is Gly.

13. A method as claimed in claim 12 wherein the nucleotide sequence of the codon encoding the amino acid is be assessed

14. A method as claimed in claim 13 wherein the nucleotide position corresponding to ORF30 2254 and\or 2279 of the EHV-1 V592 is assessed.

15. A method as claimed in claim 14 wherein the presence of a ‘G’ at position 2254 and\or 2279 is correlated with higher virulence of the herpesvirus.

16. A method as claimed in claim 15 wherein the presence of a ‘G’ at position 2254 is correlated with higher virulence.

17. A method as claimed in claim 6 which comprises the steps of:
(i) providing a sample of nucleic acid from the herpesvirus isolate, and,
(ii) establishing the presence or identity of the marker in the nucleic acid sample.

18. A method as claimed in claim 17 wherein the isolate is harboured as a latent virus within host cells, wherein said host cells are lysed to release nucleic acid molecules.

19. A method as claimed in claim 17 which comprises the step of amplifying a portion of the sample of nucleic acid containing the marker prior to determining the presence or absence of the marker variant.

20. A method as claimed in claim 19, which method comprises the steps of:
(i) providing a sample of nucleic acid from the herpesvirus isolate, and,
(b) contacting the nucleic acid molecules with oligonucleotide primers under suitable conditions permitting hybridization of the oligonucleotides to the nucleic acid molecules;
wherein a first oligonucleotide selectively binds the nucleic acid on the 3′ side of the marker, and a second oligonucleotide binds to nucleic acid on the 5′ side of the marker,
(c) enzymatically amplifying a specific region of the nucleic acid molecules comprising the marker sequence using said oligonucleotides as primers;
(d) determining whether the identity of the marker variant in the amplified sequences.

21. A method as claimed in claim 20 wherein at least one primer is a mutant oligonucleotide primer which introduces a mutation into the amplification product, such that a restriction site is created when one of the marker variants is present but not when another marker variant is present.

22. A method as claimed in claim 17 which comprises determining the binding of an oligonucleotide probe to the nucleic acid sample or amplification product therefrom, which probe comprises a nucleic acid sequence which binds specifically to one marker variant and does not bind specifically to other marker variant.

23. A method as claimed in claim 17 wherein the presence or identity of the marker variant in the nucleic acid sample is established by single strand conformation polymorphism analysis (SSCP); heteroduplex analysis; or RFLP analysis.

24. A method as claimed in claim 17 wherein the marker variant is confirmed by nucleotide sequencing.

25. A pair of oligonucleotide primers for use in probing or amplification of nucleotide positions 2254 and\or 2279 of the EHV-1 or EHV-4 ORF30-ml region, said primers being between about 18 and 30 nucleotides in length, which pair consists of a first oligonucleotide which selectively binds to nucleic acid on the 3′ side of the region and a second oligonucleotide which selectively binds to the 5′ side of the region such as to be capable of amplifying a region of between 30 to 600 nucleotides

26. A pair of oligonucleotide primers as claimed in claim 25 which amplify at least the entire ORF30-ml region.

27. A pair of oligonucleotide primers for use in probing or amplification of a region comprising any two or more of nucleotide positions 336, 344, 629, 710, 713, 719, 731-740, 755 of EHV-1 ORF68, said primers being between about 18 and 30 nucleotides in length, which pair consists of a first oligonucleotide which selectively binds to nucleic acid on the 3′ side of the region and a second oligonucleotide which selectively binds to the 5′ side of the region such as to be capable of amplifying a region of between 30 to 600 nucleotides

28. An oligonucleotide primer selected from the group consisting of the primers shown in SEQ. ANNEX 2.

29. A primer as claimed in claim 28 which is listed as ORF30f, ORF30r or ORF30s.

30. A primer as claimed in claim 28 which is listed as ORF68f, ORF68r, ORF68s1, ORF68s2 or ORF68s3.

31. A kit for assessing the virulence of herpesviruses, the kit containing a pair of oligonucleotide primers as claimed in claim 25, said kit further including one or more of the following: the reaction buffer for the respective method of enzymatic amplification, plus one or more oligonucleotides specific for an EHV marker labeled with a detectable moiety.

32. A kit for assessing the virulence of herpesviruses, the kit containing a pair of oligonucleotide primers as claimed in claim 26, said kit further including one or more of the following: the reaction buffer for the respective method of enzymatic amplification, plus one or more oligonucleotides specific for an EHV marker labeled with a detectable moiety.

33. A kit for typing EHV-1 isolates, the kit containing a pair of oligonucleotide primers as claimed in claim 27, said kit further including one or more of the following: the reaction buffer for the respective method of enzymatic amplification, plus one or more oligonucleotides specific for an EHV marker labeled with a detectable moiety.

34. A method for preparing a recombinant herpesvirus vaccine strain, which method includes the steps of:
(i) providing nucleic acid from a herpesvirus genome,
(ii) modifying the DNA POL (ORF30) gene of the herpesvirus to reduce the virulence of the gene product of said gene, wherein said modification is selected from the group consisting of insertions, substitutions, and deletion of one or more virulence markers within the region of the gene corresponding to the ORF30-ml region of Equine herpesvirus type 1 (EHV-1) V592, and
(iii) combining the modified virus encoded by the genome with a pharmaceutically acceptable diluent, adjuvant, or carrier,

35. A method as claimed in claim 34 wherein modification provides a non-acidic amino acid at the position corresponding to position 752 and/or an acidic amino acid at the position corresponding to position 760 of the region.

36. A recombinant herpesvirus strain obtainable by the method of claim 34.

37. A live viral vaccine comprising an immunogenically effective amount of the herpesvirus strain of claim 36 in a pharmaceutically acceptable carrier.

38. A vaccine as claimed in claim 37 in dosage unit form, said dosage unit being adapted to provide from 103 to 108 TCID₅₀ of the recombinant herpesvirus strain per host.

39. A method for immunizing a host against a herpesvirus disease which method includes a step of inoculating the host with an immunity-inducing dose of a vaccine as claimed in claim 37.

40. A method for immunizing a host against a herpesvirus disease which method includes a step of inoculating the host with an immunity-inducing dose of a vaccine as claimed in claim 38.

41. An isolated peptide comprising a contiguous portion of at least 10, 15, 20, 30, 40, or 50 amino acids of the amino acid sequence of the ORF30 sequence of an Equine herpesvirus type 1 (EHV-1) isolate, wherein the peptide includes at least a portion corresponding to positions 752-760 of SEQ. ANNEX 3.

42. Use of the sequence of the Equine herpesvirus type 1 (EHV-1) strain V592 polymerase ORF30-ml region in the provision of a genetic marker for assessing the virulence of a herpesvirus, which method comprises:
(i) comparing the sequence of said region with the corresponding sequence in a virulent herpesvirus strain,
(ii) identifying marker identities which differ between said sequences,
(iii) selecting from said differing marker identities, a marker which is present in said virulent herpesvirus strain sequence, but does not co-segregate with markers indicative of the sub-type or identity of the virulent herpesvirus strain.
